(19) United States
(12) Patent Application Publication
(10) Pub. No.: US 2004/0120853 A1
(43) Pub. Date: Jun. 24, 2004

(54) Biocidal control in recovery of oil by water injection

(76) Inventors: Joel F. Carpenter, Baton Rouge, LA (US); Christopher J. Nalepa, Zachary, LA (US)

Correspondence Address: EDGAR SPIELMAN, ALBEMARLE CORPORATION, 451 FLORIDA BLVD., BATON ROUGE, LA, 70801, US

(21) Appl. No.: 10/327,563

(22) Filed: Dec. 20, 2002

(51) Int. Cl. A61L 2/16; E21B 21/14

(52) U.S. Cl. 422/37; 507/277; 507/920; 166/305.1; 166/311

(57) ABSTRACT

The invention provides in a water injection system and in a water injection process for secondary oil and/or gas recovery, the presence and use of a biocidally-effective amount of a sulfamate-stabilized, bromine-based biocide with injection water that is to be used in the system or process such that bromine-based biocide is present in at least a portion of the system and/or in at least a portion of the water in the system. A composition especially adapted for use in secondary oil recovery operations, is comprised of seawater with which has been blended a biocidally-effective amount of a sulfamate-stabilized, bromine-based biocide.

### REFERENCE TO COMMONLY-OWNED RELATED APPLICATION

Commonly-owned application Ser. No.10/138,664, filed May 3, 2002, all disclosure of which is incorporated herein by reference, relates to microbiological control in oil or gas field operations.

### TECHNICAL FIELD

This invention relates to new, improved processes for effecting biocidal activity in connection with recovery of oil by injection of water, especially seawater, into the well to displace the oil toward a production location. The invention also relates to new, improved seawater compositions that provide effective biocidal activity in such oil recovery operations.

### BACKGROUND

Water injection systems are commonly used in secondary oil field recovery operations. As noted in U.S. Pat. No. 4,507,212, undesired growth of microorganisms in oil-bearing formations has plagued oil producers since the advent of water flooding as a secondary oil production technique. For example, bacterial growth can result in souring of the crude oil in a reservoir, which is caused by the reduction of inorganic sulfate compounds to sulfides by certain bacteria. If such growth is substantial, plugging of the reservoir, wells, and related equipment can occur. In addition, equipment will quickly corrode if the metal is exposed to byproducts of microbial metabolism, particularly hydrogen sulfide.

The foregoing patent further notes that although several types of microorganisms are potentially deleterious to oil production, the major problems are caused by anaerobic sulfate-reducing bacteria, especially those of the genus Desulfovibrio. For further discussions of this topic reference is made in the patent to “The Role of Bacteria in the Corrosion of Oil Field Equipment”, National Association of Corrosion Engineers, Technical Practices Committee, Pub. No. 3 (1976); Smith, R. S., and Thurlow, M. T., Guidelines Help Counter SRB Activity in Injection Water, The Oil and Gas Journal, Dec. 4, 1978, (pp 87-91); and Ruseska, I, et al., “Biocide Testing Against Corrosion-Causing Oil-field Bacteria Helps Control Plugging”, Oil and Gas Journal, Mar. 8, 1982, (pp 253-64). According to the patent, these sources generally recommend the use of a chemical microbiocide as part of a program to limit the growth of bacteria in oil fields or injection water.

As is further noted in the above patent, microorganisms in oilfields or in injection water are generally classified by their effect. Sulfate-reducing bacteria, slime-forming bacteria, iron-oxidizing bacteria, and miscellaneous organisms such as algae, sulfide oxidizing bacteria, yeast and molds, and protozoa can be encountered in bodies of water of oilfields to be sanitized.

As further pointed out in U.S. Pat. No. 4,507,212, all such microorganisms are capable of clogging filters and injection wells, and some can cause plugging of the rock formation if they can survive the temperatures and pressures found in the reservoir. In addition, certain organisms can liberate sulfide compounds which cause souring of the oil and corrosion of the wellpipe and other equipment. Unless precautions are taken to inhibit microbial growth, water flooding can seriously diminish the value of the remaining crude oil.

In U.S. Pat. No. 4,620,595 several fairly early references dealing with seawater injection in secondary recovery of oil are discussed as follows: “As indicated in ‘How to Treat Seawater for Injection Projects’ by D. L. Carlberg in World Oil, July 1979, page 67, ‘With careful treatment the virtually unlimited supply of readily available ocean water can be used successfully as a source of injection fluid for offshore or near shore pressure maintenance of water flood projects.’ The article mentions that organic growths in seawater range from bacteria to sea weed, barnacles and fish, and indicates that a basic treatment scheme, for seawater to be used as an injection medium, includes adding a biocide, filtering and deoxygenating and possibly, scale inhibiting the seawater.”

An article by R. W. Mitchell in Journal of Petroleum Technology, June 1978, page 887, is titled “The Forties Field Seawater Injection System”. The article recommends similar basic treatments of the seawater. It also describes a particular advantage of using chlorine or a hypochlorite as a biocide in combination with deoxygenation by stripping with production gas and addition of ammonium bisulfite, where the final pH of the water is about 7.5 to 9. The article mentions that although few scavengers can reduce the oxygen to less than 50 ppm, this can be achieved by bisulfite, but only if chlorine is not present in the system.

An article by C. C. McCune in Journal of Petroleum Technology, October 1982, at page 2265, is titled “Seawater Injection Experience: An Overview”. It mentions that seawater is being used more and more as the water injected into subterranean reservoirs and recommends substantially the same basic treatments of the seawater. It also indicates that adding chlorine as a biocide and SO₂ as an oxygen scavenger tends to reduce the pH of the seawater from a normal of about 8 to about 5.8.

Offshore oil recovery systems are thus highly susceptible to growth of sulfate-reducing bacteria. The presence of such bacteria and the various problems resulting from their presence can and typically do occur in various locations within such oil recovery systems. Portions of oil recovery systems where sulfate-reducing bacteria can proliferate with adverse consequences are located (i) upstream of the deacrator, (ii) from deaerator to wellheads, and (iii) downstream of wellheads. Exacerbating the situation is the ability of certain sulfate-reducing bacterial species such as Desulfovibrio desulfuricans to develop as biofilms within these portions of the oil recovery system.

While biocide compositions are available that provide biocidal activity in seawater injection systems and operations, further improvements in performance are desired. For example, a way of providing long lasting residual biocidal activity using smaller amounts of biocidal agent would be of considerable advantage. It would be especially advantageous if the biocidal agent is compatible with other components used in such operations, is relatively non-corrosive to metals, is capable of providing rapid microbiocidal activity promptly upon reaching the various loci of the microorganisms being challenged, and is effective against a variety of aerobic and anaerobic bacterial species including sulfate-reducing species that produce hydrogen sulfide and resultant “souring” of the hole.

### BRIEF SUMMARY OF THE INVENTION

This invention enables the achievement of most, if not all, of the above desirable advantages in a highly cost-effective manner.

Provided by this invention is an improvement in a water injection system and, alternatively, in a water injection process, wherein the improvement comprises effecting biocidal activity in the system and in the water being used in said system, which process comprises blending with the water a biocidally-effective amount of a sulfamate-stabilized, bromine-based biocide. Preferably, the biocide is formed from (A) a halogen source which is (i) bromine chloride, (ii) bromine and chlorine, (iii) bromine, or (iv) a mixture of any two or more of (i), (ii), and (iii), (B) a source of sulfamate anions, (C) alkali metal base, and (D) water, in amounts that the biocide composition has an active bromine content of at least 50,000 ppm, and an atom ratio of nitrogen to active bromine originating from (A) and (B) that is greater than about 0.93. Instead of using such a liquid concentrate as the biocidal agent, a biocidally-effective amount of a solid state biocidal composition formed by removal of the water from a sulfamate stabilized, bromine-based biocide can be added to or blended with the water pursuant to this invention. It is also possible to use as the sulfamate stabilized, bromine-based biocide in a given water injection system or in a given water injection process the combination of (1) a liquid concentrate as described herein and (2) a solid state biocidal agent as described herein. The water used in the water injection system and, alternatively, in the water injection process can be ordinary water (e.g., ground water or surface water such as from lakes, rivers, or streams) or it can be seawater, depending upon the location of the secondary oil recovery system or installation. Because seawater contains nutrients for bacteria thus causing greater bacterial proliferation than occurs with ordinary water, it is preferred to utilize the biocidal compositions of this invention in seawater so as to control such bacteria.

Also provided by this invention is a composition for use in a seawater injection system, which composition is comprised of seawater with which has been blended a biocidally-effective amount of an aqueous sulfamate-stabilized, bromine-based biocide. In preferred compositions of this invention, the biocide is formed from (A) a halogen source which is (i) bromine chloride, (ii) bromine and chlorine, (iii) bromine, or (iv) a mixture of any two or more of (i), (ii), and (iii), (B) a source of sulfamate anions, (C) alkali metal base, and (D) water, in amounts that the biocide composition has an active bromine content of at least 50,000 ppm and preferably at least 100,000 ppm, and an atom ratio of nitrogen to active bromine originating from (A) and (B) that is greater than about 0.93, and preferably greater than 1. In further preferred embodiments, the composition is comprised of seawater with which has been blended a biocidally-effective amount of a solid state biocidal composition formed by removal of the water from such a sulfamate-stabilized, bromine-based biocide. In other preferred embodiments, the composition is comprised of seawater with which has been blended a biocidally-effective amount of both such components, namely (1) an aqueous sulfamate-stabilized, bromine-based biocide as described herein, and (2) a solid state biocidal composition formed by removal of the water from such an aqueous sulfamate-stabilized, bromine-based biocide, the total of the individual amounts of (1) and (2) constituting the biocidally effective amount. As noted above, seawater contains nutrients which engender growth and proliferation of bacteria, and thus seawater constitutes a medium that can exacerbate the problems caused by the presence of bacteria in water injection systems operated on seawater. Provision and use of the seawater compositions of this invention thus constitute efficient and highly effective ways of minimizing the severity of such problems.

Preferred biocides are those in which the halogen source is bromine chloride, bromine and chlorine, or a mixture of bromine chloride and bromine, and the alkali metal base is a sodium or potassium base. More preferred biocides are those wherein the halogen source consists essentially of bromine chloride, wherein the alkali metal base is a sodium base, wherein the active bromine content of the biocide composition is at least 100,000 ppm, the above atom ratio of nitrogen to active bromine originating from (A) and (B) is at least about 1, and the pH of the biocide composition is at least about 12. Particularly preferred biocides are those wherein the halogen source consists essentially of bromine chloride, wherein the alkali metal base is sodium hydroxide, wherein the active bromine content of the biocide composition is at least 140,000 ppm, the above atom ratio of nitrogen to active bromine originating from (A) and (B) is at least about 1.1, and the pH of the biocide is at least about 13.

Also more preferred aqueous biocides for use in this invention are highly concentrated aqueous sulfamate-stabilized active bromine compositions which are solids-free aqueous solutions or solids-containing slurries formed as above, and in which the content of dissolved active bromine is greater than about 160,000 ppm. In the preferred aqueous solutions of this type, the active bromine in these preferred liquid biocides is all in solution at room temperature (e.g., 23° C.). In one particularly preferred embodiment the content of active bromine in such aqueous biocidal solutions (whether formed from use of (a) BrCl, or (b) Br₂, or (c) BrCl and Br₂, or (d) Br₂ and Cl₂, or (e) BrCl, Br₂ and Cl₂) is in the range of about 176,000 ppm to about 190,000 ppm (wt/wt). In another particularly preferred embodiment the content of active bromine in such aqueous biocidal solutions (whether formed from use of (a) BrCl, or (b) Br₂, or (c) BrCl and Br₂, or (d) Br₂ and Cl₂, or (e) BrCl, Br₂ and Cl₂) is in the range of from about 201,000 ppm to about 215,000 ppm.

Also preferred for use in this invention is a solid state bromine-containing biocidal composition formed by removal of water from an aqueous solution or slurry of a product formed in water from (I) a halogen source which is (i) bromine, (ii) bromine chloride, (iii) a mixture of bromine chloride and bromine, (iv) bromine and chlorine in a Br₂ to Cl₂ molar ratio of at least about 1, or (v) bromine chloride, bromine, and chlorine in proportions such that the total Br₂ to Cl₂ molar ratio is at least about 1; and (II) a source of overbased sulfamate which is (i) an alkali metal salt of sulfamic acid and/or sulfamic acid, and (ii) an alkali metal base, wherein said aqueous solution or slurry has a pH of at least 7, preferably above 10 and more preferably above 12, and an atom ratio of nitrogen to active bromine from (I) and (II) of greater than 0.93. The concentration of the product formed in water from (I) and (II) used in forming the solid state bromine-containing biocidal composition is not critical; any concentration can be present in the initial aqueous solution or slurry. Naturally it is desirable to start with a more concentrated solution or slurry as this lessens the amount of water that must be removed when preparing the solid state bromine-containing biocidal composition.

The solid state bromine-containing biocidal compositions of this invention are preferably formed by spray drying the aqueous solution or slurry of the product formed from (I) and (II) above. Temperatures of the atmosphere (e.g., dry air or nitrogen) into which the spray is directed is typically in the range of about 20 to about 100° C., and preferably is in the range of about 20 to about 60° C., particularly when the process is carried out at reduced pressure. When spray drying is used it is preferred to use the product formed from (I) and (II) as a solution rather than as a slurry as this minimizes the possibility of nozzle pluggage. On the other hand, if the water is to be flashed off or otherwise distilled from the solution or slurry of the product formed from (I) and (II), it is preferred to use the product formed from (I) and (II) as a slurry rather than as a solution as this minimizes the amount of water to be removed. Such flashing or distillations can be, and preferably are, conducted at reduced pressures to reduce the temperatures to which the product formed from (I) and (II) is exposed during drying.

The solid state bromine-containing biocidal compositions of this invention are typically in the form of powders or relatively small particles. However the solid state bromine-containing biocidal compositions of this invention can be compacted into larger forms such as nuggets, granules, pellets, tablets, pucks, and the like, by use of known procedures. Such compacted products may be formed with the use of binding agents or other materials that cause the particles to adhere one to another. If the binder used is not readily soluble in water, it is important not to totally encapsulate the product with a water-impervious coating of such binder that remains intact under actual use conditions, as this would prevent contact between the encapsulated bromine-containing biocidal composition and the water being treated with the biocidal composition. Low melting waxes or the like may be used to bind and even to encapsulate the bromine-containing biocidal composition in cases where the encapsulated product is used in waters at high enough temperatures to melt off the coating and bindings so that the water can come into contact with the previously encased biocidal composition itself. However, use of binding substances that are water-soluble or that provide effective binding action in proportions insufficient to encapsulate the particles being bound together, is preferable. The binding agent used should be compatible with the solid state bromine-containing biocidal composition of this invention.

Other aspects and embodiments of this invention will become still further apparent from the ensuing description and appended claims.

### BRIEF DESCRIPTION OF THE DRAWING

FIG. 1 is block flow diagram of a typical water injection system, illustrating various locations where, pursuant to this invention, the biocides can be fed into the system.

### GLOSSARY

The following terms as used herein have the following meanings:

activity—This term describes the amount of oxidant available for microbiological control; the term is generally used to describe the amount of active material on a percentage (or ppm) basis in given formulation. Thus, for example, a solution that contains 15% of a particular biocidal species would be said to contain 15% active ingredient or 15% active, or 150,000 ppm active ingredient.

active bromine—This term denotes the amount of oxidant available in a bromine-based biocide formulation available for microbiological control expressed relative to Br₂. Active bromine can be determined by several methods, for example, by the total bromine method described hereinafter.

biocidal activity—This term means discernable destruction of microbiological life.

biocidally-effective amount—This term denotes that the amount used controls, kills, or otherwise reduces the bacterial or microbial content of the aqueous fluid in question by a statistically significant amount as compared to the same aqueous fluid prior to treatment with a biocide of this invention.

bromonium ion—This term is used to describe bromine species in aqueous solution which have a formal positive charge and are capable of being microbiologically active. This is in contrast to bromide ion which has a formal negative charge and is not microbiologically active.

free bromine—This term is used to describe the free or relatively fast-reacting forms of bromine oxidants present in aqueous solutions. It is typically determined by performing the DPD method for free chlorine residual and multiplying the result by the conversion factor of 2.25.

ppm—This abbreviation means parts per million (wt/wt), unless specifically stated otherwise herein.

residual—The amount of oxidant in a fluid present at a given time after the oxidant has reacted with reactive impurities or components of the fluid.

total bromine—This term is used to describe both combined (relatively slow-reacting forms) and free (relatively fast-reacting) bromine oxidants present in aqueous solutions. It is typically determined by performing the DPD method for total chlorine residual and multiplying the result by the conversion factor of 2.25. This test can be used to determine “activity” or “active bromine” as described above.

seawater—any saline solution derived from the sea or other natural saline body of water, that is used in any water injection operation conducted in a system for the recovery of subterranean oil or gas whether conducted offshore or on land.

### FURTHER DETAILED DESCRIPTION OF THE INVENTION

Among the distinct advantages of this invention is that the biocides used therein, especially those made using (i) bromine chloride, (ii) a mixture of bromine chloride and bromine, (iii) bromine and chlorine in a bromine:chlorine mole ratio of greater than 1, or (iv) a combination of any two or more of (i), (ii), and (iii) as the bromine source can be effectively used to overcome bacterial problems in water injection systems and processes, especially seawater injection systems and processes, in all relevant sites including parts of the system upstream of the deaerator, from deaerator to wellheads, and downstream of wellheads.

Accordingly, seawater treated with a biocide pursuant to this invention can be used to effectively challenge bacteria and biofilm in such upstream parts of the system as lift pumps, coarse filters, and heat exchangers. It is convenient to inject such treated seawater at the lift pumps. Both aerobic and anaerobic bacteria, including sulfate-reducing bacteria, which can accumulate in these parts of the system can thereby be effectively controlled. Such accumulations of bacteria can become acute because of the plethora of nutrients normally present in seawater. If such bacterial growth becomes extensive in these upstream parts of the seawater injection system, contamination throughout the remainder of the overall seawater injection system can, and often does, occur. Moreover, temperature increases in the heat exchangers can enhance the growth of the bacteria present upstream of the deaerator and thus exacerbate the problem.

In the portions of the seawater injection system from deaerator to wellheads there are a number of potential trouble spots for bacterial growth and attendant problems. These portions include the deaerator tower, residence tanks, fine filters, and flowlines. In the deaerator tower itself where oxygen is removed from the seawater and an oxygen scavenger is employed to assist in this operation, residual biocide introduced upstream is typically destroyed. Therefore, pursuant to this invention an effective biocidal amount of a sulfamate-stabilized bromine-based biocide as described herein is introduced into the deaerated seawater downstream of the deaerator tower. The addition site for such biocide should be proximate to the exit side of the deaerator tower. Bacteria can also accumulate in the residence tanks which are locations well-suited for such accumulation to occur. Because the seawater has been degassed and usually treated with an oxygen scavenger, the conditions in the residence tanks are anaerobic and thus highly conducive to the development and growth of sulfate-reducing bacteria. Another factor enhancing bacterial growth in the residence tanks is the elevated temperature condition within the tanks. Thus, pursuant to this invention a sufficient amount of biocidal agent utilized pursuant to this invention is caused to be present in the seawater entering the residence tanks. In this way, the development and growth of the bacteria, including sulfate-reducing bacteria, can be effectively challenged. Fine filters which are typically present between the deaerator and wellheads have a tendency of collecting and thereby enhancing the growth of bacteria on their surfaces. Thus, the seawater treated with a biocide pursuant to this invention when passing through the fine filters and contacting the filter surfaces, effectively controls such bacterial concentration and growth on such surfaces. Despite the fact that the injected seawater passes through the flowlines, the interior walls of the flowlines constitute additional sites for bacterial growth and attachment. Biofilm development has been known to become excessive on these interior walls. However, pursuant to this invention, the seawater passing through such flowlines contains a sufficient amount of the biocide such that such growth and attachment is substantially reduced, if not eliminated. In this regard the powerful biocidal action exerted by the biocides used pursuant to this invention is especially effective in the control of biofilm growth and development.

Bacterial contamination in the parts of the water injection system downstream of wellheads is also of concern, and can be effectively controlled pursuant to this invention. The presence and accumulation of bacteria downstream of the wellheads typically results from carry-off from bacterial accumulations in low-flow or stagnant portions of the system proximate to the wellheads, such as in downhole safety valves and in deadleg zones of downhole tubing. The active biocidal content in the seawater present in the system from a biocide used pursuant to this invention can effectively control the bacterial accumulations, including biofilms, that normally tend to form in the injection system downstream of wellheads.

Thus in accordance with this invention problems normally caused by bacterial growth and accumulation in various portions of the water injection system as well as in the well formation itself can be effectively controlled by use in the water being used in the system of a biocidally effective amount of a sulfamate-stabilized active bromine composition utilized pursuant to this invention. Among the problems that are effectively reduced, if not eliminated, by this invention are (A) excessive corrosion, especially of mild steel, in the injection system which may be attributed at least in part to acidic conditions fostered by sulfate-reducing bacteria, (B) pluggage in the injection system due to accumulation of bacteria and/or biofilms on filters or in valves and the like, and (C) damage to the reservoir itself such as (i) pluggage in the formation which may result at least in part from deposition of particulate matter from corrosion or resulting from the action of surfactants used in the system and/or souring of the formation which can be attributed at least in part to the action of sulfate-reducing bacteria.

Some of the biocide compositions used in the practice of this invention are known. Methods for the preparation of the known compositions are given, for example, in U.S. Pat. Nos. 3,558,503; 6,068,861; 6,110,387; 6,299,909; 6,306,441; and 6,322,822. The solid state bromine-containing biocidal compositions referred to above and some highly concentrated aqueous solutions or slurries are novel compositions that are also described in detail in commonly-owned copending application Ser. No. 10/282,290, filed Oct. 28, 2002, all disclosure of which is incorporated herein by reference. Such highly concentrated solutions and slurries include the following:

A) An aqueous biocide composition comprising a water solution or slurry having in in solution therein (i) an active bromine content derived from (a) bromine chloride, or (b) bromine, or (c) bromine chloride and bromine, or (d) bromine and chlorine, or (e) bromine chloride, bromine, and chlorine, of greater than about 160,000 ppm (wt/wt), and (ii) an overbased alkali metal salt of sulfamic acid (most preferably a sodium salt), and optionally containing—but preferably containing—(iii) an alkali metal halide (preferably sodium chloride or sodium bromide, or both), wherein the relative proportions of (i) and (ii) are such that the atom ratio of nitrogen to active bromine is greater than 0.93, and preferably is greater than 1 (e.g., in the range of above 1 to about 1.5) and wherein the pH of the composition is at least 7 (e.g., in the range of 10 to about 13.5, and preferably in the range of about 12.5 to about 13.5, or even as high as about 14). The content of active bromine in these solutions is typically in the range of above 160,000 ppm to about 215,000 ppm. Preferably, the content of active bromine in these concentrated liquid biocidal solutions (whether formed from use of (a) BrCl, or (b) Br₂, or (c) BrCl and Br₂, or (d) Br₂ and Cl₂), or (e) BrCl, Br₂ and Cl₂), is in the range of about 165,000 ppm (wt/wt) to about 215,000 ppm (wt/wt), more preferably in the range of about 170,000 ppm (wt/wt) to about 215,000 ppm (wt/wt), and still more preferably in the range of about 176,000 ppm (wt/wt) to about 215,000 ppm (wt/wt).

B) A composition as in A) immediately above wherein the content of active bromine in the concentrated liquid biocidal compositions (whether formed from use of (a) BrCl, or (b) Br₂, or (c) BrCl and Br₂, or (d) Br₂ and Cl₂, or (e) BrCl, Br₂ and Cl₂) is in the range of about 176,000 ppm to about 190,000 ppm (wt/wt).

C) A composition as in A) immediately above wherein the content of active bromine in the liquid biocidal compositions (whether formed from use of (a) BrCl, or (b) Br₂, or (c) BrCl and Br₂, or (d) Br₂ and Cl₂, or (e) BrCl, Br₂ and Cl₂) is in the range of from about 201,000 ppm to about 215,000 ppm.

While biocides made by use of bromine can be used (e.g., U.S. Pat. No.3,558,503) as the sulfamate stabilized, bromine-based biocides of this invention, preferred biocides of this invention because of their effectiveness and stability are formed from bromine chloride, bromine and chlorine, or a mixture of bromine chloride and up to about 50 mole % of bromine. A particularly preferred biocide of this type for use in the practice of this invention is commercially available from Albemarle Corporation under the trademark WELLGUARD™ 7030 biocide. The sulfamate used in the production of such biocide products is effective in stabilizing the active bromine species over long periods of time, especially when the pH of the product is at least about 12 and preferably at least about 13. For example, WELLGUARD™ 7030 biocide is stable for greater than one year if protected from sunlight. For ease of reference, these preferred highly effective and highly stable aqueous biocides for use in the practice of this invention formed from bromine chloride, bromine and chlorine, or a mixture of bromine chloride and up to about 50 mole % of bromine, a sulfamate source such as sulfamic acid or sodium sulfamate, a sodium base, typically NaOH, and water are often referred to hereinafter collectively as “preferred aqueous biocides” or “the preferred aqueous biocides”, and in the singular as “preferred aqueous biocide” or “the preferred aqueous biocide”.

Another commercially-available biocide solution containing sulfamate stabilizer and which can be used as the sulfamate stabilized, bromine-based biocide in the practice of this invention is Stabrex™ biocide (Nalco Chemical Company).

The blending operation can be conducted in any manner conventionally used in blending additives into water used in water injection systems. Since the many of the biocides, including the preferred biocides, whether formed on site or received from a manufacturer, are mobile aqueous solutions, the blending is rapid and facile. Simple metering or measuring devices and means for mixing or stirring the biocide with the water to be used in the system can thus be used, if desired. Periodically individual batches of such water, typically seawater, can be treated with the biocide and used so that the biocide is provided intermittently to the well being flooded, i.e., the well into which water, especially seawater, is being injected. Preferably, however, all of the water used in a given operation is treated with a biocide of this invention so that the biocide is continuously being provided to the well being flooded.

The solid state bromine-containing biocidal compositions referred to above are water soluble powders or particulate solids, and are easily blended with the water being used in the water injection system. For example, the solids can be poured or metered into the water at one or more suitable locations upstream from the appropriate point(s) at which the so-treated water enters into the injection system.

Typically the amount of the biocide used should provide in the range of about 1 to about 10 ppm, and preferably in the range of about 2 to about 6 ppm of active bromine species in the blended water prior to injection into the system. Departures from these ranges whenever deemed necessary or desirable are permissible and are within the scope of this invention.

Some components or impurities commonly encountered in or by aqueous injection fluids are reactive with the biocides used pursuant to this invention. One such impurity is, as noted above, hydrogen sulfide. Another such impurity is oil, particularly hydrocarbonaceous oil. Such components are identifiable as substances which are reactive in aqueous media with monobromo alkali metal sulfamate, dibromo alkali metal sulfamate, or bromonium ions. When such components are present, their presence can be overcome provided the quantity of such components can be effectively overcome by use of a sacrificial quantity of a biocide used pursuant to this invention. In wells that have recently been drilled or serviced, residual amounts of guar, polyacrylamide, scale inhibitor, and various other additives or components of well fluids used in the drilling or servicing may be encountered. Many such common well fluid components are surprisingly compatible with biocides employed in the practice and compositions of this invention. Starch, on the other hand, is an example of a potential well fluid component which is not necessarily compatible with biocides of this invention. The presence of starch and like components in the well may, however, be overcome using a sacrificial quantity of the biocide.

One of the advantages of using the preferred biocides is their great compatibility with other components used in downhole operations. For example, unlike HOBr and hypobromites, the preferred biocides do not oxidize or otherwise destroy organic phosphonates typically used as corrosion and scale inhibitors. In fact, the preferred biocides are compatible with residual components of both gel-type and slickwater-type fracturing fluids as long as they are devoid or substantially devoid of hydrogen sulfide. Hydrogen sulfide can react rapidly with the biocides used pursuant to this invention, including the preferred biocides. Therefore, if there is some hydrogen sulfide present in the aqueous drilling fluid, it is preferred to determine analytically the amount of hydrogen sulfide that is present in the downhole solution. If the amount is sufficiently small that it does not require an excessive amount of the biocide to consume that amount of hydrogen sulfide, the amount of the biocide present in seawater injected into the well should be sufficient not only to consume the hydrogen sulfide but additionally to provide a suitable residual quantity of active bromine in the well. Since at least the preferred biocides are highly cost-effective, it is economically feasible to sacrifice some of the biocide as a means of destroying the hydrogen sulfide so that the remainder of the biocide injected can provide the appropriate residual of active bromine in the well being flooded. Of course if the amount of hydrogen sulfide is so high as to make it non-feasible economically to destroy the hydrogen sulfide using the biocide, the use of the compositions of this invention in such well is not recommended. The dividing line as between how much hydrogen sulfide can be tolerated and consumed with extra biocide pursuant to this invention and how much makes it non-feasible to do so will vary depending upon a number of variable economic factors as well as technical factors. For example, such factors as operating costs, well location, particular biocide being used, degree of bacterial infestation, and the amount of active bromine residual needed or desired can have a significant effect upon how much hydrogen sulfide can be tolerated in any given situation. Therefore, the amount of hydrogen sulfide that can be tolerated and overcome in the downhole aqueous fluid pursuant to this invention is subject to considerable latitude and cannot be universally quantified. Suffice it to say that the well being treated should either be free of hydrogen sulfide or may contain in the downhole aqueous fluid a “consumable amount” of hydrogen sulfide. The “consumable amount” of hydrogen sulfide that can be tolerated can be, and should be, determined on a small scale experimentally before conducting a full scale operation. As a general guide, it has been found that application of 50 ppm of WELLGUARD 7030 biocide solution (thereby theoretically yielding 7.5 ppm residual as Br₂) provided about 2 ppm residual as Br₂ going downhole. In the presence of 5 ppm of hydrogen sulfide, it would take about 300 ppm of WELLGUARD 7030 biocide solution, i.e., about 45 ppm of biocide (100% active basis) to react with the hydrogen sulfide. To establish a suitable measurable residual, an additional amount in the range of about 10 to about 200 ppm, e.g., about 50 ppm of the WELLGUARD 7030 biocide solution should be added. The presence of 5 ppm hydrogen sulfide thus increases the WELLGUARD 7030 biocide solution application rate from about 50 ppm to about 350 ppm. On the basis of present-day economic conditions it is estimated that the maximum consumable amount of hydrogen sulfide in the aqueous fluid is about 10 ppm. Thus in the future, this estimated value should be escalated upwardly or downwardly in proportion to the change in the consumer price index.

As is known in the art, aqueous well fluids can contain various additive components such as clay, bentonite, and other colloidal materials; weighting agents such as barium sulfate, amorphous silica, calcium carbonate, and hematite; preservatives such as formaldehyde, sodium trichlorophenate, and sodium pentachlorophenate; fluid loss control agents such as carboxymethyl cellulose, corn meal, silica flour, or starch; viscosity modifying agents such as ferrochrome lignosulfonate, calcium lignosulfonate, or sodium lignosulfonate; emulsifiers; surfactants; and the like.

In the case of aqueous gel-type fracturing fluids various gelation agents and crosslinking agents are used. Examples of gelation agents include guar gum, derivatized guar gums such as hydroxypropyl guar, xanthan gums, cellulosic materials such as carboxymethylhydroxyethyl cellulose and hydroxyethyl cellulose, and similar materials. Guar gum is a commonly used gelation agent. Typical crosslinkers used include borates, chromates, titanates, zirconates, aluminates, and antimony crosslinking agents. Slickwater-type fracturing fluids typically contain a viscosity modifying or viscosity reducing agent. Oftentimes a low molecular weight water-soluble polymeric material serves as a viscosity reducing agent in slickwater fluids. Among additives of this type are polyacrylamide, acrylic acid homopolymers, copolymers of maleic acid and sulfonated styrene, copolymers of acrylic or methacrylic acid and a water-soluble salt of allyl or methallyl sulfonic acid or the like. Polyacrylamide-type slickifier additives are commonly used.

Besides providing persistent and long lasting residual biocidal activity, e.g., providing a measurable residual lasting for a period of at least one hour and typically at least 2 hours in the seawater being injected into the well, the preferred biocides also provide very rapid biocidal activity upon coming in contact with the downhole microorganisms. Usually, extensive bacterial “knockdown” occurs within an hour or two. Consequently, measurements of effective residual biocidal activity can be taken within two to three hours after injection of the seawater treated with biocide pursuant to this invention to thereby ensure that a sufficient amount of biocidally-effective species has been injected into the well. Thus usage of the seawater treated pursuant to this invention can shorten and simplify the water injection and oil recovery operations.

The rapid bacterial “knockdown” (e.g., 1 or more log reduction of bacteria in one hour) activity achievable by the practice of this invention is surprising in view of the fact that the biocides are stabilized compositions by virtue of their sulfamate content. In short, despite their great stability, the preferred biocides function unexpectedly quickly.

Another advantage of the preferred biocides is that they are highly effective against a wide variety of heterotrophic bacteria, of both the aerobic and anaerobic types. Moreover, sulfate-reducing bacterial species are effectively controlled or killed by use of the preferred biocides. This in turn can eliminate, or at least greatly diminish, the generation of hydrogen sulfide which normally is produced as a product of bacterial reduction of sulfates, and thereby prevent the well from turning sour.

Still another advantage of this invention is the very low corrosivity of the preferred biocides against metals, especially ferrous metals. This is the result of the low oxidation-reduction potential of the preferred biocides.

Yet another advantage of this invention is the stability of at least the preferred biocides at elevated temperatures. Thus unlike HOBr or hypobromite solutions which have relatively poor thermal stability at elevated temperatures, the preferred biocides can be used in very deep wells where highly elevated temperatures are encountered without premature decomposition. This in turn provides the means for effectively combating heat resistant bacteria that reside at such deep locations.

Standard analytical test procedures are available enabling close approximation of “total bromine” and “free bromine” present in aqueous solution. For historical and customer familiarity reasons, these procedures actually express the results of the determinations as “free chlorine” and “total chlorine”, which results can then be arithmetically converted to “total bromine” and “free bromine”. The procedures are based on classical test procedures devised by Palin in 1974. See A. T. Palin, “Analytical Control of Water Disinfection With Special Reference to Differential DPD Methods For Chlorine, Chlorine Dioxide, Bromine, Iodine and Ozone”, J. Inst. Water Eng., 1974, 28, 139. While there are various modernized versions of the Palin procedures, the version of the tests for “free chlorine” and “total chlorine” recommended herein for use, are fully described in Hach Water Analysis Handbook, 3rd edition, copyright 1997. The procedure for “free chlorine” is identified in that publication as Method 8021 appearing on page 335, whereas the procedure for “total chlorine” is Method 8167 appearing at page 379. Briefly, the “free chlorine” test involves introducing to the halogenated water a powder comprising DPD indicator powder and a buffer. “Free chlorine” present in the water reacts with the DPD indicator to produce a red to pink coloration. The intensity of the coloration depends upon the concentration of “free chlorine” species present in the sample. This intensity is measured by a calorimeter calibrated to transform the intensity reading into a “free chlorine” value in terms of mg/L Cl₂. Similarly, the “total chlorine” test also involves use of DPD indicator and buffer. In this case, KI is present with the DPD and buffer whereby the halogen species present, including nitrogen-combined halogen, reacts with KI to yield iodine species which turn the DPD indicator to red/pink. The intensity of this coloration depends upon the sum of the “free chlorine” species and all other halogen species present in the sample. Consequently, this coloration is transformed by the calorimeter into a “total chlorine” value expressed as mg/L Cl₂.

In greater detail, these procedures are as follows:

1. To determine the amount of species present in the aqueous well fluid water which respond to the “free chlorine” and “total chlorine” tests, the sample should be analyzed within a few minutes of being taken, and preferably immediately upon being taken.

2. Hach Method 8021 for testing the amount of species present in the sample which respond to the “free chlorine” test involves use of the Hach Model DR 2010 calorimeter or equivalent. The stored program number for chlorine determinations is recalled by keying in “80” on the keyboard, followed by setting the absorbance wavelength to 530 nm by rotating the dial on the side of the instrument. Two identical sample cells are filled to the 10 mL mark with the aqueous sample under investigation. One of the cells is arbitrarily chosen to be the blank. Using the 10 mL cell riser, this is admitted to the sample compartment of the Hach Model DR 2010, and the shield is closed to prevent stray light effects. Then the ZERO key is depressed. After a few seconds, the display registers 0.00 mg/L Cl₂. To a second cell, the contents of a DPD Free Chlorine Powder Pillow are added. This is shaken for 10-20 seconds to mix, as the development of a pink-red color indicates the presence of species in the sample which respond positively to the DPD test reagent. Within one minute of adding the DPD “free chlorine” reagent to the 10 mL of aqueous sample in the sample cell, the blank cell used to zero the instrument is removed from the cell compartment of the Hach Model DR 2010 and replaced with the test sample to which the DPD “free chlorine” test reagent was added. The light shield is then closed as was done for the blank, and the READ key is depressed. The result, in mg/L Cl₂ is shown on the display within a few seconds. This is the “free chlorine” level of the water sample under investigation.

3. Hach Method 8167 for testing the amount of species present in the aqueous sample which respond to the “total chlorine” test involves use of the Hach Model DR 2010 calorimeter or equivalent. The stored program number for chlorine determinations is recalled by keying in “80” on the keyboard, followed by setting the absorbance wavelength to 530 nm by rotating the dial on the side of the instrument. Two identical sample cells are filled to the 10 mL mark with the water under investigation. One of the cells is arbitrarily chosen to be the blank. To the second cell, the contents of a DPD Total Chlorine Powder Pillow are added. This is shaken for 10-20 seconds to mix, as the development of a pink-red color indicates the presence of species in the water which respond positively to the DPD “total chlorine” test reagent. On the keypad, the SHIFT TIMER keys are depressed to commence a three-minute reaction time. After three minutes the instrument beeps to signal the reaction is complete. Using the 10 mL cell riser, the blank sample cell is admitted to the sample compartment of the Hach Model DR 2010, and the shield is closed to prevent stray light effects. Then the “ZERO” key is depressed. After a few seconds, the display registers 0.00 mg/L Cl₂. Then, the blank sample cell used to zero the instrument is removed from the cell compartment of the Hach Model DR 2010 and replaced with the test sample to which the DPD “total chlorine” test reagent was added. The light shield is then closed as was done for the blank, and the READ key is depressed. The result, in mg/L Cl₂ is shown on the display within a few seconds. This is the “total chlorine” level of the water sample under investigation.

4. To convert the readings to bromine readings, the “free chlorine” and the “total chlorine” values should be multiplied by 2.25 to provide the “free bromine” and the “total bromine” values.

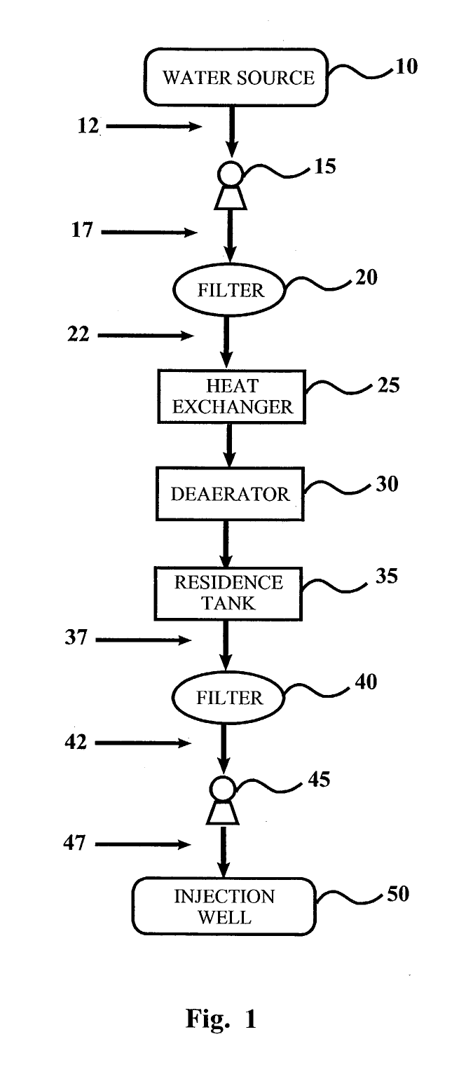

FIG. 1 of the Drawing illustrates schematically the flow paths in a typical water injection system for secondary recovery of oil and/or gas. It will be appreciated that more than one unit referred to in the depicted system may be in the system, that one or more of the units referred to in the depicted system may be omitted or replaced by equivalent apparatus, and that suitable variations in the flowpath shown may be utilized in a given system. Referring now to the Drawing, in the system depicted lift pump 15 takes water, typically seawater, from water source 10 and transmits the water to filter 20 which typically is a coarse filter designed to remove sand and other solid debris from the water. The cleansed water from filter 20 is then passed into and through heat exchanger 25, which is used to adjust the temperature of the water to a suitable temperature typically in the range of about 10 to about 40° C. and preferably in the range of about 20 to about 30° C., and thence into deaerator apparatus 30 such as one or more deaerator towers. After removal of the air from the water it then is passed into residence tank 35. Water from residence tank 35 is passed through filter 40 which typically is designed to remove entrained fine particles from the water. In systems where corrosion has occurred, such fine particles may include particles of rust and/or other corrosion products, as well as fine particles initially present in water source 10. Pump 45 transmits the filtered water under pressure into the injection well 50. Pursuant to this invention, one or more biocidal compositions referred to herein can be fed into the system at various locations. Thus a suitable biocidal quantity of a biocide can be introduced into the water as it is picked up from source 10 and before entering pump 15, as indicated by arrow 12. Instead, or in addition, the biocide or additional biocide can be fed between pump 15 and filter 20 as indicated by arrow 17. Other illustrative locations for feeds, or supplemental feeds, are shown as arrows 22, 37, 42, and 47. It is not necessary to feed at each location depicted, nor is it necessary that the concentration of biocide fed at one location be the same as the concentration at another location. And it is not required that the biocides of this invention be the same at different feed locations of a given system. For example a more concentrated biocide of the invention can be fed at one location and a less concentrated biocide of the invention at another location. Similarly, a solution of a biocide of the invention can be fed at one location and a solid state biocide of the invention can be fed at another location. Because of the effectiveness of the biocides of the invention, these are now matters within the discretion of the operator and to some extent will depend on the tendencies for microbial growth to occur at various locations in a given system, as well as the type of microbial growth that may be encountered in any given system under the prevailing operating conditions being used for the system. In general it is desirable to ensure that a feed of a biocidal quantity of the biocide into the water occur upstream of any location where undesirable microbial growth and accumulation may occur, and thus at least a feed as at 12 or 17 is preferred so as to minimize corrosion and microbial growth and accumulation in the lines and apparatus of the system contacted by the incoming water. This is especially important in the case of seawater because of its large nutrient content which typically enhances microbial growth and accumulation throughout the system. It is also preferred to introduce additional biocide downstream of the deaerator especially at 37 so that microbial growth and accumulation does not clog filter 40 or cause excessive corrosion in the downstream portions of the system contacted by the water. Also some degradation of the biocide may occur within the deaerator. To combat downhole bacteria such as sulfate-reducing bacteria, it is often desirable to make a further feed of biocide at 42 or 47 so that fresh biocide is available to provide downhole biocidal activity.

It can be seen that the system depicted in FIG. 1 comprises deaerator 30; a section upstream from the deaerator composed of lift pump 15, filter 20, heat exchanger 25, and lines for water flow through this upstream section from water source 10 to deaerator 30; and a section from deaerator to wellhead composed of residence tank 35, filter 40, pump 45, and lines for water flow through this downstream section from the deaerator to the wellhead. The section downstream of the wellhead, though not depicted, is composed of apparatus known to those of ordinary skill in the art.

The following Examples are presented for purposes of illustration, and are not intended to unduly limit the scope of this invention. Examples 1-5 serve to illustrate, in downhole operations other than water injection systems or operations, the advantageous properties of biocidal compositions used pursuant to this invention.

In Examples 1-3 a group of experiments was conducted on a laboratory scale using WELLGUARD 7030 biocide (Albemarle Corporation) as the biocide composition to demonstrate the powerful biocidal activity that such a product exhibits in aqueous media. In these experiments a typical gel-type fracturing fluid was formulated by initial preparation of a 500 g sample of WELLGUARD 7030 biocide at a bromine residual level of 100 or 30 ppm in synthetic water and then addition of the various fracturing fluid components. The 100 and 30 ppm bromine levels correspond to product application rates of 667 or 200 ppm, respectively. The decay in the halogen residual was monitored at regular time intervals. A control formulation was also prepared at 30 ppm bromine residual level by adding WELLGUARD 7030 biocide to relatively demand-free synthetic water.

In particular, the activity of the WELLGUARD 7030 biocide being used was 10.8% or 108,000 ppm as BrCl (15.0% or 150,000 ppm as Br₂). Chemicals used in forming the gel-type fracturing fluid consisted of PLEXSURF WRS (surfactant), CLAYMAX (clay-control agent), PLEXGEL 907L (oil suspension of guar gum), and PLEXBOR 97 (crosslinker). The chemical used for the slickifier-type fracturing fluid work was PLEXSLICK 961 (anionic polyacrylamide suspension). CELITE 545 filter aid and Gelman ACRODISC 5 μm syringe filters (Gelman part #4489) were employed for clarifying some solutions prior to DPD analysis in the gel-type fracturing fluid studies. Microbiological supplies were obtained from several sources. PetriFilm aerobic count plates and Butterfield's buffer (used for serial dilutions) were obtained from Edge Biologicals (Memphis, Tenn.). SRB broth bottles were obtained from C&S Laboratories Inc. (Broken Arrow, Okla.).

A sample of synthetic water (SW) was prepared by adding CaCl₂ (0.91 g), NaHCO₃ (0.71 g) and NaCl (0.10 g) to one gallon of deionized water. The sample contained about 50 ppm alkalinity (as CaCO₃), 100 ppm calcium hardness (as CaCO₃), and 150 ppm chloride. The pH was 8.1.

A stock solution of WELLGUARD 7030 biocide was prepared by diluting 1.35 g WELLGUARD 7030 biocide to 200 g with synthetic water. Analysis by the DPD method indicated the activity of the stock solution was 993 ppm as Br₂ (i.e., 0.511 g of stock was diluted to 125.0 g with deionized water; the Hach DPD reading was 4.06 ppm after 3 minutes).

The general procedure used for preparing the fracturing fluids involved adding the following components in the following order to a 1-liter stainless steel blending cup:

1) Appropriate amounts of WELLGUARD 7030 biocide stock solution and synthetic water for 500 g total solution.

2) PLEXSURF WRS surfactant (0.5 mL).

3) CLAYMAX clay-control agent (0.5 mL).

4) PLEXGEL 907L guar gum (3.75 mL)

This mixture was stirred at 1100 rpm for 10 minutes to disperse the additives. In some cases PLEXBOR 97 crosslinking agent (0.6 mL) was then added to the stirred mixture whereby the mixture thickened immediately. This mixture was then stirred for an additional 2-3 minutes at about 1100 rpm. All samples were diluted 1:20 with deionized water and mixed for 2 minutes with a magnetic stirrer. The total halogen residual (as Br₂) was measured using a Hach DR/2000 spectrophotometer. An optional procedure for removing haziness for more accurate residual analysis involved adding 0.3 g Celite 545 filter aid and stirring. The mixture was then filtered through a 5.0 micron Gelman ACRODISC syringe filter directly into a 10 mL Hach cuvette for DPD analysis.

### EXAMPLE 1

### Determination of Bromine Residual Persistency in a Gel-Type Fracturing Fluid Using WELLGUARD 7030 Biocide at 100 ppm as Br₂

A kitchen blender with a one-liter stainless steel cup was charged with WELLGUARD 7030 biocide stock solution (50.5 g) and synthetic water (449.5 g). This provided an initial bromine residual of 100 ppm as Br₂ or 670 ppm as applied product. Reagents were added as indicated above. Samples were then analyzed at regular intervals by performing 1:20 dilutions of gel in deionized water and stirring vigorously with a magnetic stirrer to disperse most of the gel into the solutions. The hazy solution was then analyzed by the DPD method.

### EXAMPLE 2

### Determination of Bromine Residual Persistency in a Gel-Type Fracturing Fluid Using WELLGUARD 7030 Biocide at 30 ppm as Br₂

The procedure of Example 1 was used except that the amount of the WELLGUARD 7030 biocide stock solution used was 15.15 g and the amount of synthetic water used was 484.85 g. This provided an initial bromine residual of 30 ppm as Br₂ or 200 ppm as applied product.

### EXAMPLE 3

### Control Run Using WELLGUARD 7030 Biocide in Synthetic Water at 30 ppm as Br₂

For control purposes, WELLGUARD 7030 biocide 15.15 g was added to synthetic water (484.85 g). The sample was diluted 1:20 in deionized water and analyzed by the Hach method.

In Examples 1 and 2, it was found that after 15 minutes, the halogen residual retention was about 30%. This remained at 20% after 2 hours and about 6% after 18 hours. It was subsequently found that because of difficulties in sample workup (the stirring speed used was found to be much too slow), the residual bromine results obtained in Examples 1 and 2 were lower than the actual amounts of residual bromine present. Nevertheless, these results show that the preferred biocides provide suitably long-lasting bromine residuals. In addition, it was found that the properties of the gel were unaffected by the biocide treatment.

A field study was conducted on use of WELLGUARD 7030 biocide in a slickwater fracturing fluid. One part of this study involved determining the bromine residual of the slickwater fracturing fluid. Another part of this study involved determining the microbiological effects of the preferred biocides in such fracturing fluids. These studies are referred to in Examples 4 and 5, respectively.

### EXAMPLE 4

### Analysis of Pit Water with Slickwater Additives and a Preferred Biocide

At a fracturing site in Texas, a sample of the pit water to be used for the fracturing job was sampled. The pit water looked relatively clean. The water was treated with a conventional slickifier additive. The water after treatment was only slightly hazy. WELLGUARD 7030 biocide was added to this water to provide a theoretical 7.5 ppm bromine residual (50 ppm based on applied product solution) and the activity was measured immediately after mixing and after a period of 15 minutes. The activity was 1.41 ppm (after mixing) and 1.38 ppm (after 15 minutes). These results indicated that at a 50 ppm treatment level as applied product, it is possible to get a measurable and long-term residual with this pit water formulated with slickwater additives.

### EXAMPLE 5

### Microbiological Tests of Pit Water With Slickwater Additives and a Preferred Biocide Additive

In these experiments microbiological tests were performed by conducting serial dilutions using Butterfield's buffer and plating 1 mL onto PetriFilm aerobic count plates. Pit water was the water source used for the job and was contained in a plastic-lined pond located about 300 yards from the job site. This water was pumped to a series of mix tanks. From there, the water was formulated with Plexslick 961, WELLGUARD 7030 biocide, and sand. Three diesel-powered pumps rated at 2240 HP each provided the power to drive the mixture downhole into the formation at a rate of 3000 gpm and a pressure of about 3000 psi. Experiments with the pit water indicated some demand relative to bottled water. The slickwater additive introduced additional demand. The “pit water+additives” study was performed by pulling a sample of pit water, adding the slickwater agent (Plexslick 961) and then introducing WELLGUARD 7030 biocide at a 7.5 ppm level as bromine. This experiment indicates that treatment at 50 ppm applied product affords a measurable and long-term residual in this pit water formulated with slickwater additives. Work was also performed on the water in the mix tanks. This “mix water” was rust-colored and had been standing in contact with the metal container, and thus probably represented a worst case in terms of microbiological activity for the water to be used for the fracturing job. Finally, analysis of the formulated slickwater at the job site (“frac job water”) indicated that the desired bromine residual was achieved and that it was persistent. Microbiological data indicate low bacteria counts and a 3-log reduction from levels present in the mix water. The results of this field study are summarized in the Table 1.

TABLE 1
Field Study: WELLGUARD 7030 Biocide Treatment of a
Slickwater Fraccing Formulation
(WELLGUARD 7030 Biocide Addition at 50 ppm as Product
or Equivalent)

| | Biocide | Br₂ Residual | | Microbiocidal |
|---|---|---|---|---|
| | Contact | Theoretical, | Actual, | Results |
| Sample | Time | ppm | ppm | Aerobic, CFU/mL |
| Pit Water | Before | — | — | 6.4 × 10³ |
| Pit Water | Initial | 7.5 | 4.2 | — |
| Pit Water | 15 mins. | 7.5 | 3.8 | — |
| Pit Water + | Initial | 7.5 | 1.4 | — |
| Additives¹ | | | | |
| Pit Water + | 15 mins. | 7.5 | 1.4 | — |
| Additives | | | | |
| Mix Water | Before | — | — | 1.1 × 10⁵ |
| Frac Job Water² | Initial | 7.5 | 2.3 | 2.0 × 10³ |
| Frac Job Water² | 30 mins. | 7.5 | 1.6 | 5.2 × 10¹ |
| Frac Job Water² | 1 hr. | 7.5 | — | 6.1 × 10¹ |

¹Additives are Plexslick 961 and WELLGUARD 7030 biocide.
²Frac job water was sampled about 1 hour into the job. It consists of water from the mix tank (mix water) plus additives.

The studies of Examples 1-5 demonstrate that the preferred biocides exemplified by WELLGUARD 7030 biocide were compatible with the gel-type and slickwater-type fracturing fluids. The laboratory experiments in a guar-based gel-type fracturing formulation indicate that the preferred biocide, WELLGUARD 7030 biocide, provided a persistent and long-lasting residual. Properties of the gel were unaffected by treatment with the biocide. The field study in the slickwater-type fracturing job demonstrated that WELLGUARD 7030 biocide applied at 50 ppm as product provided a 3-log reduction in aerobic bacteria counts. This job used a polyacrylamide-based formulation.

Another important finding from the foregoing field test was that one drum of WELLGUARD 7030 biocide (˜600 lbs) treated the entire 1.1 million gallons of formulated slickwater. This fracturing job would have required 7 drums of a popular competitive biocide, THPS (tetrakishydroxymethylphosphonium sulfate). This work clearly indicates that WELLGUARD 7030 biocide can provide good knockdown of bacteria while being cost effective in oil field applications.

Example 6 illustrates the lower oxidation reduction potential and thus lower metal corrosivity of preferred biocides as compared to two other well-known halogen-containing biocides, namely bleach and activated sodium bromide.

### EXAMPLE 6

### Comparative Study of Oxidation Reduction Potentials (ORP)

The oxidants studied consisted of WELLGUARD 7030 biocide, STABREX biocide (stabilized sodium hypobromite), bleach (NaOCl), and activated sodium bromide (NaOCl and NaBr). The WELLGUARD 7030 biocide had an activity of 10.88% as BrCl or 6.69% as Cl₂. The STABREX biocide had an activity of 9.70% as BrCl or 5.96% as Cl₂. The bleach was industrial grade and had an activity of 2.42% as Cl₂.

Stock solutions of the biocides were prepared at 1000 ppm halogen residual concentration (as Cl₂) in brown glass bottles using deionized water for dilution. Solution activities were confirmed using the DPD method and a Hach Co. (Loveland, Colo.) DR/2000 spectrophotometer. Information concerning the stock solutions made and used are summarized in Table 2.

TABLE 2

| Biocide | Biocide Activity, % | Biocide, g | Deionized water, g |
|---|---|---|---|
| STABREX | 5.96 | 1.72 | 100 |
| WELLGUARD | 6.69 | 1.52 | 100 |
| 7030 biocide | | | |
| Bleach | 2.42 | 6.00 | 140 |
| Bleach + | 2.42 | 6.00 | 140 |
| NaBr | NA | 0.41 | 100 |

In Table 2 the activities of the bromine-based biocides are expressed as total halogen residual (as Cl₂); the activity of bleach is expressed as free halogen residual (as Cl₂). Activities expressed in terms of free halogen residuals for the stock solutions in Table 2 were STABREX biocide, 974 ppm; WELLGUARD 7030 biocide, 840 ppm; activated sodium bromide, 960 ppm.

Aliquots of the stock solutions above were added to 1000 mL of cooling tower water that had been pulled from a cooling tower. A 1000 mL beaker was charged with 1000 mL of cooling tower water and stirred while measuring ORP with a Brinkmann Metrohm 716 DMS Titrino automatic titrator. It took about 45 minutes for the sample to equilibrate—the ORP reading would gradually decline to a reading of about 300 mV. The sample was deemed to have equilibrated when the change in the ORP reading was less than or equal to 1 unit/minute. At this point, 0.5 g of stock solution (nominal halogen residual=0.5 ppm) was added and the mixture allowed to equilibrate once again. A sample was pulled to determine free and total halogen residuals and then 0.5 g additional stock solution was added and the process repeated. The following aliquots were added during the experiment: 0.5 g, 1.0 g, 2.0 g, 3.0 g, 4.0 g, 6.0 g, 8.0 g, 10.0 g.

The ORP data obtained from these studies are summarized in Table 3.

TABLE 3

| | Nominal | | Actual | | |
|---|---|---|---|---|---|
| | Residual, ppm | | Residual, ppm | | ORP |
| Biocide | Free | Total | Free | Total | Reading, mV |
| STABREX | 0 | 0 | ND | ND | 302 |
| | 0.49 | 0.51 | 0.41 | 0.44 | 426 |
| | 0.98 | 1.04 | 0.72 | 0.82 | 497 |
| | 2.00 | 2.11 | 1.56 | 1.73 | 560 |
| | 3.04 | 3.20 | 2.68 | 2.86 | 571 |
| | 4.09 | 4.32 | 3.88 | 4.12 | 579 |
| | 6.26 | 6.60 | 6.20 | 6.60 | 586 |
| | 8.47 | 8.94 | 8.82 | 9.24 | 593 |
| | 10.74 | 11.33 | 11.52 | 12.06 | 597 |
| WELLGUARD | 0 | 0 | ND | ND | 307 |
| 7030 biocide | | | | | |
| | 0.42 | 0.52 | 0.34 | 0.45 | 410 |
| | 0.85 | 1.04 | 0.62 | 0.83 | 487 |
| | 1.72 | 2.12 | 1.28 | 1.68 | 558 |
| | 2.62 | 3.22 | 2.22 | 2.80 | 571 |
| | 3.53 | 4.20 | 3.23 | 4.05 | 576 |
| | 5.40 | 6.63 | 5.30 | 6.60 | 583 |
| | 7.31 | 8.98 | 7.42 | 9.17 | 587 |
| | 9.26 | 11.38 | 9.90 | 11.79 | 591 |
| Bleach | 0 | | ND | ND | 339 |
| | 0.50 | | 0.13 | 0.34 | 500 |
| | 1.00 | | 0.29 | 0.48 | 620 |
| | 2.04 | | 1.12 | 1.29 | 659 |
| | 3.09 | | 1.88 | 2.08 | 672 |
| | 4.17 | | 2.98 | 3.43 | 678 |
| | 6.37 | | 5.24 | 5.68 | 683 |
| | 8.63 | | 7.68 | 8.16 | 685 |
| | 10.93 | | 10.08 | 10.78 | 689 |
| Activated NaBr | 0 | 0 | ND | ND | 297 |
| | 0.48 | 0.52 | 0.16 | 0.23 | 495 |
| | 0.97 | 1.05 | 0.30 | 0.41 | 592 |
| | 1.97 | 2.14 | 0.88 | 1.10 | 641 |
| | 2.99 | 3.25 | 1.47 | 1.85 | 670 |
| | 4.03 | 4.39 | 2.52 | 2.82 | 688 |
| | 6.17 | 6.71 | 4.62 | 4.77 | 699 |
| | 8.35 | 9.08 | 6.60 | 7.35 | 703 |
| | 10.58 | 11.51 | 8.60 | 9.50 | 710 |

It can be seen from Table 3 that WELLGUARD 7030 biocide and STABREX biocide, which represent biocides used in the practice of this invention, behaved similarly with respect to ORP response. They yielded lower ORP values compared to conventional oxidizing biocides such as bleach and activated sodium bromide. In addition both WELLGUARD 7030 biocide and STABREX biocide exhibited little loss in biocide residual under the conditions of these experiments. In contrast, bleach and activated sodium bromide underwent significant loss of residual during initial stages of biocide addition.

Example 7 illustrates the greater compatibility of preferred biocides as compared to two well-known halogen-containing biocides, namely bleach and activated sodium bromide with respect to phosphonate additives for aqueous drilling fluids.

### EXAMPLE 7

### Comparative Study of Compatibilities of Several Halogen-Containing Biocides Toward Phosphonate Additives

The oxidants studied consisted of WELLGUARD 7030 biocide, bleach (NaOCl), and activated sodium bromide (NaOCl and NaBr). The WELLGUARD 7030 biocide and bleach were added directly. Activated sodium bromide was prepared in situ by introducing 20 ppm bromide ion to the stock solution followed by addition of bleach. The phosphonates used in this work consisted of AMP (aminomethylene phosphonic acid), HEDP (hydroxyethylidene diphosphonic acid), and PBTC (phosphonobutanetricarboxylic acid). These materials were commercial samples (Mayoquest 1320, 1500, and 2100, respectively) obtained from Callaway Chemical Co. (Smyrna, Ga.).

Solutions consisting of 5 ppm scale inhibitor (as active phosphonate) in the presence of 10 ppm oxidant (as Cl₂) were prepared as follows. To 900 mL deionized water were added appropriate stock solutions containing phosphonate, alkalinity (NaHCO₃), and calcium hardness (CaCl₂). The pH was adjusted to 9.1 with 5% aq. NaOH and diluted up to 1 L in a dark amber bottle. A dose of oxidant was added to achieve a residual of 10 ppm. The solutions were then periodically monitored for phosphonate reversion by determining the reversion to orthophosphate (Hach method 490). The oxidant residual was also periodically monitored using the DPD method (Hach method 80). All of this work was performed at room temperature (23° C.). The initial active phosphonate content was confirmed by conversion to orthophosphate via UV/persulfate oxidation followed by a conventional phosphate analysis (Hach method 501). A conversion factor was applied to the phosphate measurement to determine the initial amount of active phosphonate present as follows: AMP, 1.05; HEDP, 1.085; PBTC, 2.85.

The experimental data for the effect of the various biocides on AMP, HEDP, and PBTC are presented in Tables 4, 5, and 6, respectively.

TABLE 4
Effect of Oxidizing Biocides on Reversion of AMP
to Orthophosphate

| Time, | | WELLGUARD | Activated | |
|---|---|---|---|---|
| minutes | Analysis, ppm | 7030 biocide | NaBr | Bleach |
| 0 | Phosphate | 4.58¹ | 4.18¹ | 4.22¹ |
| 0 | Active Phosphonate² | 4.8 | 4.4 | 4.4 |
| 20 | Phosphate | 0.36 | 0.82 | 0.35 |
| 40 | Phosphate | 0.22 | 0.99 | 0.7 |
| 70 | Phosphate | 0.16 | 1.1 | 0.53 |
| 100 | Phosphate | 0.36 | 1.27 | 0.75 |
| 130 | Phosphate | 0.24 | 1.36 | 0.8 |
| 190 | Phosphate | — | 1.15 | 0.77 |
| 220 | Phosphate | 0.36 | 1.07 | 0.59 |
| 250 | Phosphate | 0.33 | 1.2 | 0.64 |
| 280 | Phosphate | 0.32 | 1.08 | 0.83 |
| 310 | Phosphate | 0.32 | 1.12 | 0.82 |
| 340 | Phosphate | 0.32 | 1.15 | 0.8 |
| 370 | Phosphate | 0.32 | 1.13 | 0.81 |
| 400 | Phosphate | 0.35 | 1.22 | 0.79 |
| 460 | Cl₂ | 10.2 | 8.6 | 9.4 |
| 520 | Phosphate | 0.3 | 1.31 | 0.97 |
| 1360 | Phosphate | 0.47 | 0.88 | 0.91 |
| 100-1360 | Phosphate (average) | 0.34 | 1.16 | 0.79 |

¹Maximum amount of ortho-phosphate that can be liberated (determined by UV/persulfate oxidation of AMP, Hach method 501).
²Phosphate analysis X conversion factor (=1.05).

TABLE 5
Effect of Oxidizing Biocides on Reversion of HEDP
to Orthophosphate

| Time, | | WELLGUARD | | |
|---|---|---|---|---|
| minutes | Analysis, ppm | 7030 biocide | Activated NaBr | Bleach |
| 0 | Phosphate | 4.20¹ | 4.40¹ | 4.80¹ |
| 0 | active phosphonate² | 4.6 | 4.8 | 5.2 |
| 20 | Phosphate | 0.24 | 0.67 | 0 |
| 40 | Phosphate | 0.01 | 1.69 | 0 |
| 70 | Phosphate | 0.05 | 1.93 | 0.2 |
| 100 | Phosphate | 0.08 | 1.96 | 0.25 |
| 130 | Phosphate | 0.12 | 2.11 | 0.31 |
| 190 | Phosphate | 0.21 | 2.58 | 0.61 |
| 220 | Phosphate | 0.24 | 2.55 | 0.65 |
| 250 | Phosphate | 0.18 | 2.63 | 0.39 |
| 280 | Phosphate | 0.2 | 2.66 | 0.41 |
| 310 | Phosphate | 0.3 | 2.71 | 0.58 |
| 340 | Phosphate | 0.39 | 2.75 | 0.65 |
| 370 | Phosphate | 0.35 | 2.25 | 0.84 |
| 400 | Phosphate | 0.33 | 2.34 | 0.65 |
| 400 | Cl₂ | 10.5 | 6.85 | 10.6 |
| 460 | Phosphate | 0.37 | 2.37 | 0.95 |
| 520 | Phosphate | 0.5 | 2.75 | 0.94 |

¹Maximum amount of ortho-phosphate that can be liberated (determined by UV/persulfate oxidation of AMP, Hach method 501).
²Phosphate analysis X conversion factor (=1.085).

TABLE 6
Effect of Oxidizing Biocides on Reversion of PBTC
to Orthophosphate

| Time, | | WELLGUARD | Activated | |
|---|---|---|---|---|
| minutes | Analysis, ppm | 7030 biocide | NaBr | Bleach |
| 0 | Phosphate | 1.72¹ | 1.82¹ | 1.44¹ |
| 0 | active phosphonate² | 4.9 | 5.2 | 4.1 |
| 30 | Phosphate | 0 | 0 | 0 |
| 60 | Phosphate | 0 | 0 | 0 |
| 90 | Phosphate | 0 | 0 | 0 |
| 120 | Phosphate | 0 | 0 | 0 |
| 150 | Phosphate | 0 | 0 | 0 |
| 180 | Phosphate | 0 | 0 | 0 |
| 210 | Phosphate | 0 | 0.38 | 0.12 |
| 270 | Phosphate | 0.2 | 0.24 | 0.16 |
| 330 | Phosphate | 0.08 | 0.04 | 0.05 |
| 360 | Phosphate | 0.06 | 0.17 | 0.02 |
| 390 | Phosphate | 0.09 | 0.01 | 0.02 |
| 390 | Phosphate | 8.75 | 9.6 | 9.5 |
| 1360 | Phosphate | 0.06 | 0.02 | 0.08 |
| 210-1360 | Phosphate, average | 0.082 | 0.142 | 0.075 |

¹Maximum amount of ortho-phosphate that can be liberated (determined by UV/persulfate oxidation of AMP, Hach method 501).
²Phosphate analysis X conversion factor (=2.85).

The data in Table 4 show that WELLGUARD 7030 biocide, a preferred biocide, is less aggressive towards AMP than either bleach and activated sodium bromide toward amino methylene phosphonic acid (AMP), a common phosphonate additive. The relative order is:

WELLGUARD 7030 biocide<bleach<activated sodium bromide

Although there is some scatter in the data, phosphonate reversion remained essentially unchanged with all biocides within 100 minutes of reaction time. The averaged amounts of phosphonate reversion were 7.4% (WELLGUARD 7030 biocide), 18.7% (bleach), and 27.8% (activated sodium bromide).

The data in Table 5 show that WELLGUARD 7030 biocide is also less aggressive toward hydroxyethylidene diphosphonic acid (HEDP), another common phosphonate additive than the other two biocides tested. In fact, HEDP is significantly less stable in the presence of activated sodium bromide than both bleach or WELLGUARD 7030 biocide. Phosphonate reversion appeared to increase regularly with time with all biocides although again there is some scatter in the data. The relative amounts of reversion after 520 minutes were 11.9% (WELLGUARD 7030 biocide), 19.6% (bleach), and 62.5% (activated sodium bromide).

From the data in Table 6 it can be seen that none of the biocides was particularly aggressive towards phosphonobutanetricarboxylic acid (PBTC). In fact no phosphonate reversion was detected with any biocide until 3½ hours of contact. The average amounts of phosphonate reversion after 3½ hours contact and beyond were 4.8% (WELLGUARD 7030 biocide), 5.2% (bleach), and 7.8% (activated sodium bromide).

It is evident from the results summarized in Tables 4, 5, and 6, that WELLGUARD 7030 biocide used pursuant to this invention is significantly less aggressive to commonly used phosphonates in comparison to bleach and activated sodium bromide. This in turn indicates that at least the preferred biocides used pursuant to this invention offer increased compatibility with potential well fluid component additives as compared to bleach and activated sodium bromide.

Example 8 illustrates the efficacy of the biocides of the invention in seawater, especially in combating sulfate-reducing bacteria.

### EXAMPLE 8

Samples from two random lots of WELLGUARD 7030 biocide were subjected to tests conducted substantially in accordance with the Official Methods of Analysis of AOAC International, 17th Edition, 2000 Chapter 6, Disinfectants Section 965.13. Each lot of test substance was tested in triplicate at 10 ppm, measured as bromine, in Instant Ocean salt solution prepared with “chlorine demand free” water against the respective test organisms, Desulfovibrio desulfuricans subsp. desulfuricans, ATCC 7757, Bacillus cereus, ATCC 11778, and Pseudomonas fluorescens, ATCC 13525. Instant Ocean synthetic sea salt is available from Aquarium Systems, Inc., Mentor, Ohio. A dilution/aliquot of the test material was brought into contact with a known population of test bacteria for a specified period of time. A sample was then plated to enumerate the surviving bacteria. The log₁₀ survivors and log₁₀ reduction from the original population were calculated. The exposure conditions were 10 minutes, 1 hour, 3 hours and 24 hours for Desulfovibrio desulfuricans and 10 minutes. 1 hour and 3 hours for Bacillus cereus and Pseudomonas fluorescens at 20±1° C. The average log₁₀ survivors and the average log₁₀ reduction in numbers of bacteria, compared to an untreated control, were calculated for each time point for both lots of WELLGUARD 7030 biocide. The test results are summarized in Table 7.

It can be seen that at 10 ppm bromine and with a 10 minute exposure time, a >3 log₁₀ reduction in numbers of test bacteria was shown with both lots of WELLGUARD 7030 biocide against Desulfovibrio desulfuricans subsp. desulfuricans, ATCC 7757.

Under the same test conditions, with up to 3 hours of exposure, no reduction in numbers of Pseudomonas fluorescens, ATCC 13525 was seen and ˜0.3 log₁₀ reduction in numbers of Bacillus cereus, ATCC 11778 was seen for both lots of STABROM® 909 Biocide.

### SUMMARY TABLE OF RESULTS—LOG₁₀ REDUCTION

### Summary of Results for STABROM® 909 @10 ppm Bromine Diluted in ½ Cup/Gal “Instant Ocean”

TABLE 7

| | | | | D. desulfuricans | | | |
|---|---|---|---|---|---|---|---|
| | | B. cereus, | | subsp. desulfluricans, | | P. fluorescens, | |
| | | ATCC 11778 | | ATCC 7757 | | ATCC 13525 | |
| | | *Log₁₀ | | Log₁₀ | | Log₁₀ | |
| Sample | | Survivor | **Log₁₀ | Survivor | Log₁₀ | Survivor | Log₁₀ |
| Id./Exposure | | s/mL | Reduction | s/mL | Reduction | s/mL | Reduction |
| 8525-66-1 | 10 MIN. | 6.04 | 0.11 | <2.00 | >3.00 | 4.91 | NR |
| MDV-99-2 | | 6.08 | 0.07 | <2.00 | >3.00 | 4.84 | NR |
| 8525-66-1 | 1 hour | 5.91 | 0.24 | <2.00 | >3.00 | 4.84 | NR |
| MDV-99-2 | | 6.00 | 0.15 | <2.00 | >3.00 | 4.91 | NR |
| 8525-66-1 | 3 hour | 5.88 | 0.27 | <2.00 | >3.00 | 4.84 | NR |
| MDV-99-2 | | 5.82 | 0.33 | <2.00 | >3.00 | 4.77 | NR |
| 8525-66-1 | 24 hour | NT | NT | <2.00 | >3.00 | NT | NT |
| MDV-99-2 | | NT | NT | <2.00 | >3.00 | NT | NT |

| Untreated | | | | | | |
|---|---|---|---|---|---|---|
| Numbers Control | | | | | | |
| CFU/mL | Log₁₀/mL | CFU/mL | Log₁₀/mL | CFU/mL | Log₁₀/mL | CFU/mL |
| | 6.15 | 1.4 × 10⁶ | ˜5.00 | ˜1.0 × 10⁵ | 4.73 | 5.4 × 10⁴ |

NR = No Reduction
NT = Not Tested
*Log₁₀ of CFU/mL (average of three replicate tests)
**Reduction as compared to untreated numbers control

Compounds referred to by chemical name or formula anywhere in this document, whether referred to in the singular or plural, are identified as they exist prior to coming into contact with another substance referred to by chemical name or chemical type (e.g., another component, a solvent, or etc.). It matters not what preliminary chemical changes, if any, take place in the resulting mixture or solution, as such changes are the natural result of bringing the specified substances together under the conditions called for pursuant to this disclosure. Also, even though the claims may refer to substances in the present tense (e.g., “comprises”, “is”, etc.), the reference is to the substance as it exists at the time just before it is first contacted, blended or mixed with one or more other substances in accordance with the present disclosure.

Except as maybe expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

All documents referred to herein are incorporated herein by reference in toto as if fully set forth in this document.

This invention is susceptible to considerable variation in its practice. Therefore the foregoing description is not intended to limit, and should not be construed as limiting, the invention to the particular exemplifications presented hereinabove. Rather, what is intended to be covered is as set forth in the ensuing claims and the equivalents thereof permitted as a matter of law.

That which is claimed is:

1. In a water injection process in a system for secondary oil and/or gas recovery, the improvement which comprises blending a biocidally-effective amount of a sulfamate-stabilized, bromine-based biocide with injection water for use in said process such that bromine-based biocide is present in at least a portion of the system and/or in at least a portion of the water in said system.

2. The improvement as in claim 1 wherein the biocide used in said blending is an aqueous concentrate formed from (A) a halogen source which is (i) bromine chloride, (ii) bromine and chlorine, (iii) bromine, or (iv) a mixture of any two or more of (i), (ii), and (iii), (B) a source of sulfamate anions, (C) alkali metal base, and (D) water, in amounts such that the biocide has an active bromine content of at least 50,000 ppm, a pH of at least 7, and an atom ratio of nitrogen to active bromine from (A) and (B) that is greater than about 0.93.

3. The improvement as in claim 2 wherein said active bromine content is at least 100,000 ppm.

4. The improvement as in claim 2 wherein said active bromine content is above 160,000 ppm.

5. The improvement as in claim 2 wherein said active bromine content is in the range of about 176,000 ppm to about 190,000 ppm.

6. The improvement as in claim 2 wherein said active bromine content is in the range of about 201,000 ppm to about 215,000 ppm.

7. The improvement as in claim 1 wherein the biocide used in said blending is an aqueous concentrate that has a pH of at least about 12.

8. The improvement as in any of claims 2-6 wherein said aqueous concentrate has a pH of at least about 12.

9. The improvement as in claim 1 wherein the biocide used in said blending is a solid state bromine-containing biocidal composition formed by removal of water from an aqueous solution or slurry of a sulfamate-stabilized, bromine-based biocide.

10. The improvement as in claim 9 wherein the aqueous solution or slurry from which water is removed is a sulfamate-stabilized, bromine-based biocide composition formed in water from (I) a halogen source which is (i) bromine, (ii) bromine chloride, (iii) a mixture of bromine chloride and bromine, (iv) bromine and chlorine in a Br₂ to Cl₂ molar ratio of at least about 1, or (v) bromine chloride, bromine, and chlorine in proportions such that the total Br₂ to Cl₂ molar ratio is at least about 1; and (II) a source of overbased sulfamate which is (i) an alkali metal salt of sulfamic acid and/or sulfamic acid, and (ii) an alkali metal base, wherein said aqueous solution or slurry has a pH of at least 7, and an atom ratio of nitrogen to active bromine from (I) and (II) of greater than 0.93.

11. The improvement as in claim 10 wherein the pH of said aqueous solution or slurry before removal of the water therefrom is above 7, and wherein the atom ratio of nitrogen to active bromine from (I) and (II) of said aqueous solution or slurry before removal of the water therefrom is greater than 1.

12. In the operation of a water injection system for secondary oil or gas recovery wherein the system comprises a deaerator, a section upstream from the deaerator, a section from deaerator to wellhead, and a section downstream of wellhead, and wherein water is caused to flow in at least portions of each of said sections, the improvement which comprises blending with water that is caused to flow in at least a portion of at least one said section, a biocidally-effective amount of a sulfamate-stabilized, bromine-based biocide such that biocide is provided in at least a portion of said at least one said section.

13. The improvement as in claim 12 wherein water with which a biocidally-effective amount of a sulfamate-stabilized, bromine-based biocide has been blended is caused to flow in at least a portion of each of at least two said sections such that biocide is provided in at least a portion of each of said at least two said sections.

14. The improvement as in claim 12 wherein water with which a biocidally-effective amount of a sulfamate-stabilized, bromine-based biocide has been blended is caused to flow in at least a portion of each of all three of said sections such that biocide is provided in at least a portion of each of said all three said sections.

15. The improvement as in any of claims 12-14 wherein the biocide used in said blending is an aqueous concentrate formed from (A) a halogen source which is (i) bromine chloride, (ii) bromine and chlorine, (iii) bromine, or (iv) a mixture of any two or more of (i), (ii), and (iii), (B) a source of sulfamate anions, (C) alkali metal base, and (D) water, in amounts such that the concentrate has an active bromine content of at least 50,000 ppm, a pH of at least 7, and an atom ratio of nitrogen to active bromine from (A) and (B) that is greater than about 0.93.

16. The improvement as in claim 15 wherein said active bromine content is at least 100,000 ppm, wherein said atom ratio is greater than 1, and wherein said aqueous concentrate has a pH of at least about 12.

17. The improvement as in claim 15 wherein said active bromine content is above 160,000 ppm, wherein said atom ratio is greater than 1, and wherein said aqueous concentrate has a pH of at least about 12.

18. The improvement as in claim 15 wherein said active bromine content is in the range of about 176,000 ppm to about 190,000 ppm, wherein said atom ratio is greater than 1, and wherein said aqueous concentrate has a pH of at least about 12.

19. The improvement as in claim 15 wherein said active bromine content is in the range of about 201,000 ppm to about 215,000 ppm, wherein said atom ratio is greater than 1, and wherein said aqueous concentrate has a pH of at least about 12.

20. The improvement as in any of claims 12-14 wherein the biocide used in said blending is a solid state bromine-containing biocidal composition formed by removal of water from an aqueous solution or slurry of a sulfamate-stabilized, bromine-based biocide.

21. The improvement as in claim 20 wherein the aqueous solution or slurry from which water is removed is a sulfamate-stabilized, bromine-based biocide formed in water from (I) a halogen source which is (i) bromine, (ii) bromine chloride, (iii) a mixture of bromine chloride and bromine, (iv) bromine and chlorine in a Br₂ to Cl₂ molar ratio of at least about 1, or (v) bromine chloride, bromine, and chlorine in proportions such that the total Br₂ to Cl₂ molar ratio is at least about 1; and (II) a source of overbased sulfamate anion which is (i) an alkali metal salt of sulfamic acid and/or sulfamic acid, and (ii) an alkali metal base, wherein said aqueous solution or slurry has a pH of at least 7, and an atom ratio of nitrogen to active bromine from (I) and (II) of greater than 0.93.

22. The improvement as in claim 21 wherein the pH of said aqueous solution or slurry before removal of the water therefrom is above 7, and wherein the atom ratio of nitrogen to active bromine from (I) and (II) of said aqueous solution or slurry before removal of the water therefrom is greater than 1.

23. In a water injection system for secondary oil or gas recovery wherein the system comprises a deacrator, a section upstream from the deaerator, a section from deaerator to wellhead, and a section downstream of wellhead, and wherein water is caused to flow in at least portions of each of said sections, the improvement which comprises the presence in at least a portion of at least one said section of water containing a biocidally effective amount of a biocide formed by blending with said water a biocidally-effective amount of a sulfamate-stabilized, bromine-based biocide.

24. The improvement as in claim 21 wherein the sulfamate-stabilized, bromine-based biocide blended with said water is an aqueous concentrate formed from (A) a halogen source which is (i) bromine chloride, (ii) bromine and chlorine, (iii) bromine, or (iv) a mixture of any two or more of (i), (ii), and (iii), (B) a source of overbased alkali metal sulfamate, (C) alkali metal base, and (D) water, in amounts such that the concentrate has an active bromine content of at least 50,000 ppm, a pH of at least 7, and an atom ratio of nitrogen to active bromine from (A) and (B) that is greater than about 0.93.

25. A composition as in claim 24 wherein said halogen source is bromine or a mixture of bromine chloride and bromine; wherein said source of overbased sulfamate is (i) sodium sulfamate and/or sulfamic acid, and (ii) sodium hydroxide; wherein said active bromine content is at least 100,000 ppm; and wherein said aqueous concentrate has a pH of at least about 12.

26. A composition as in claim 23 wherein the sulfamate-stabilized, bromine-based biocide blended with said water is a solid state bromine-containing biocidal composition formed by removal of water from an aqueous solution or slurry of a sulfamate-stabilized, bromine-based biocide.

27. A composition as in claim 26 wherein the aqueous solution or slurry from which water is removed is a sulfamate-stabilized, bromine-based biocide composition formed in water from (I) a halogen source which is (i) bromine, (ii) bromine chloride, (iii) a mixture of bromine chloride and bromine, (iv) bromine and chlorine in a Br₂ to Cl₂ molar ratio of at least about 1, or (v) bromine chloride, bromine, and chlorine in proportions such that the total Br₂ to Cl₂ molar ratio is at least about 1; and (II) a source of overbased sulfamate which is (i) an alkali metal salt of sulfamic acid and/or sulfamic acid, and (ii) an alkali metal base, wherein said aqueous solution or slurry has a pH of at least 7, and an atom ratio of nitrogen to active bromine from (I) and (II) of greater than 0.93.

28. A composition as in claim 27 wherein said halogen source is bromine or a mixture of bromine chloride and bromine; wherein said source of overbased sulfamate is (i) sodium sulfamate and/or sulfamic acid, and (ii) sodium hydroxide; wherein said bromine-based biocide composition has an active bromine content of at least 100,000 ppm before removal of water therefrom; and wherein said bromine-based biocide composition has a pH of at least about 12 before removal of water therefrom.

29. A composition especially adapted for use in secondary oil recovery operations, which composition is comprised of seawater with which has been blended a biocidally-effective amount of a sulfamate-stabilized, bromine-based biocide.

30. A composition as in claim 29 wherein the biocide used in said blending is an aqueous concentrate formed from (A) a halogen source which is (i) bromine chloride, (ii) bromine and chlorine, (iii) bromine, or (iv) a mixture of any two or more of (i), (ii), and (iii), (B) a source of sulfamate anions, (C) alkali metal base, and (D) water, in amounts that the biocide has an active bromine content of at least 50,000 ppm, a pH of at least 7, and an atom ratio of nitrogen to active bromine from (A) and (B) that is greater than about 0.93.

31. A composition as in claim 30 wherein said active bromine content is at least 100,000 ppm, wherein said atom ratio is greater than 1, and wherein said aqueous concentrate has a pH of at least about 12.

32. A composition as in claim 30 wherein said active bromine content is above 160,000 ppm, wherein said atom ratio is greater than 1, and wherein said aqueous concentrate has a pH of at least about 12.

33. A composition as in claim 30 wherein said active bromine content is in the range of about 176,000 ppm to about 190,000 ppm, wherein said atom ratio is greater than 1, and wherein said aqueous concentrate has a pH of at least about 12.

34. A composition as in claim 30 wherein said active bromine content is in the range of about 201,000 ppm to about 215,000 ppm, wherein said atom ratio is greater than 1, and wherein said aqueous concentrate has a pH of at least about 12.

35. A composition as in claim 29 wherein the sulfamate-stabilized, bromine-based biocide is a solid state bromine-containing biocidal composition formed by removal of water from an aqueous solution or slurry of a sulfamate-stabilized, bromine-based biocide.

36. A composition as in claim 35 wherein the aqueous solution or slurry from which water is removed is a sulfamate-stabilized, bromine-based biocide formed in water from (I) a halogen source which is (i) bromine, (ii) bromine chloride, (iii) a mixture of bromine chloride and bromine, (iv) bromine and chlorine in a Br₂ to Cl₂ molar ratio of at least about 1, or (v) bromine chloride, bromine, and chlorine in proportions such that the total Br₂ to Cl₂ molar ratio is at least about 1; and (II) a source of overbased sulfamate which is (i) an alkali metal salt of sulfamic acid and/or sulfamic acid, and (ii) an alkali metal base, wherein said aqueous solution or slurry has a pH of at least 7 and an atom ratio of nitrogen to active bromine from (I) and (II) of greater than 0.93.

37. A composition as in claim 36 wherein the pH of said aqueous solution or slurry before removal of the water therefrom is above 7, and wherein the atom ratio of nitrogen to active bromine from (I) and (II) of said aqueous solution or slurry before removal of the water therefrom is greater than 1.

38. A composition as in any of claims 30, 31, 36, or 37 wherein said halogen source is bromine or a mixture of bromine chloride and bromine, and said source of overbased sulfamate is (i) sodium sulfamate and/or sulfamic acid, and (ii) sodium hydroxide.

39. In a water injection process in a system for secondary oil or gas recovery, the improvement which comprises blending a biocidally-effective amount of a sulfamate-stabilized, bromine-based biocide with injection water for use in said process such that bromine-based biocide is present in at least a portion of the system and/or in at least a portion of the water in said system.

40. A process which comprises blending a biocidally-effective amount of a sulfamate-stabilized, bromine-based biocide with seawater to form a biocidal seawater solution, and injecting the biocidal seawater solution as the water injection medium in a water injection system for secondary oil recovery such that biocidal activity is provided within at least a portion of said system.

41. A process as in claim 40 wherein said system contains sulfur-reducing bacteria.
